(19) United States
(12) Patent Application Publication
(10) Pub. No.: US 2020/0375491 A1
(43) Pub. Date: Dec. 3, 2020

(54) HEARTBEAT ANALYZING METHOD AND HEARTBEAT ANALYZING METHOD

(71) Applicants: KURA Care LLC, San Diego, CA (US); KURA Med Inc., Taipei (TW)

(72) Inventors: Kai-Chieh Yang, San Diego, CA (US); Ming-Tse Tsai, San Diego, CA (US); Minjun Chen, San Diego, CA (US); Chih-Wei Chiu, Kaohsiung City (TW); Alvin Hsu, San Diego, CA (US); Ka Tin Hui, San Diego, CA (US)

(73) Assignees: KURA Care LLC, San Diego, CA (US); KURA Med Inc., Taipei (TW)

(21) Appl. No.: 16/874,688

(22) Filed: May 15, 2020

Related U.S. Application Data
(60) Provisional application No. 62/847,958, filed on May 15, 2019.

(51) Int. Cl. A61B 5/0468; G16H 40/67; G16H 50/70; G16H 50/20; G16H 10/60; G06N 20/00; A61B 5/04; A61B 5/0456; A61B 5/00

(52) U.S. Cl. CPC A61B 5/0468; G16H 40/67; G16H 50/70; G16H 50/20; A61B 5/7267; G06N 20/00; A61B 5/04017; A61B 5/0456; A61B 5/6801; G16H 10/60

(57) ABSTRACT

A heartbeat analyzing method and a heartbeat analyzing system are provided. The heartbeat analyzing method includes: sensing a user using a wearable device and acquiring a physiological signal record; performing a dispersion calculation to the physiological signal record using the wearable device and generating a Poincaré plot of the physiological signal record; and inputting the Poincaré plot into a heart rhythm classifier model and determining a heartbeat classification of the user based on personal health data of the user.

### CROSS-REFERENCE TO RELATED APPLICATION

This application claims the priority benefit of U.S. provisional application Ser. No. 62/847,958, filed on May 15, 2019. The entirety of the above-mentioned patent application is hereby incorporated by reference herein and made a part of this specification.

### BACKGROUND

### 1. Technical Field

The disclosure generally relates to an analyzing method and an analyzing system, in particular, to a heartbeat analyzing method and a heartbeat analyzing system.

### 2. Description of Related Art

After acquiring the heartbeat signal record of a subject, most of the conventional heartbeat analyses are conducted manually to examine the heartbeat signal record based on rule of thumb, to determine whether the heart rhythm of the subject is regular or arrhythmic. Under such condition, the determination of the heart rhythm is subject to human factors, such as subjective interpretations that vary from person to person, which can lead to the inconsistency in the criteria of determination. Furthermore, different subjects have different physical conditions or personal health records. When the analysis is performed by humans, such differences may not be taken into consideration, such that the criteria of determination may not be optimally adjusted accordingly. Besides lowering the accuracy in heart rhythm determination, this may also lead to a misjudgment and may even endanger the lives of the subjects.

### SUMMARY

Accordingly, the present disclosure provides an analyzing method and an analyzing system for a user to eliminate accurately non-ideal factors when the heartbeat is determined to be regular or arrhythmic, and to further make a personalized determination of the heartbeat based on personal conditions of the user.

The heartbeat analyzing method of the disclosure includes sensing a user using a wearable device to acquire a physiological signal record, performing a dispersion calculation to the physiological signal record using the wearable device to generate a Poincaré plot of the physiological signal record, and inputting the Poincaré plot into a heart rhythm classifier model to determine a heartbeat classification of the user based on personal health data of the user.

The heartbeat analyzing system of the disclosure includes a wearable device and a host device. The wearable device senses a user to acquire a physiological signal record, and performs a dispersion calculation on the physiological signal record to generate a Poincaré plot of the physiological signal record. The host device is communicatively connected to the wearable device and stores a heart rhythm classifier model. The host device inputs the Poincaré plot and personal health data of the user into the heart rhythm classifier model to determine a heartbeat classification of the user.

Based on the above, the heartbeat analyzing method and the heartbeat analyzing device of the disclosure may eliminate the non-ideal factors in the heart rhythm determination and make an accurate personalized determination based on the personal health condition of the user, thereby lowering the possibility of making human errors and further improving efficiently the accuracy in determining whether a heart rhythm is regular or not.

### BRIEF DESCRIPTION OF THE DRAWINGS

FIG. 1 is a schematic view of a heartbeat analyzing system according to an embodiment of the disclosure.

FIG. 2A is a schematic view of a heartbeat analyzing method according to an embodiment of the disclosure.

FIG. 2B is a schematic view of a heartbeat analyzing method according to another embodiment of the disclosure.

### DESCRIPTION OF THE EMBODIMENTS

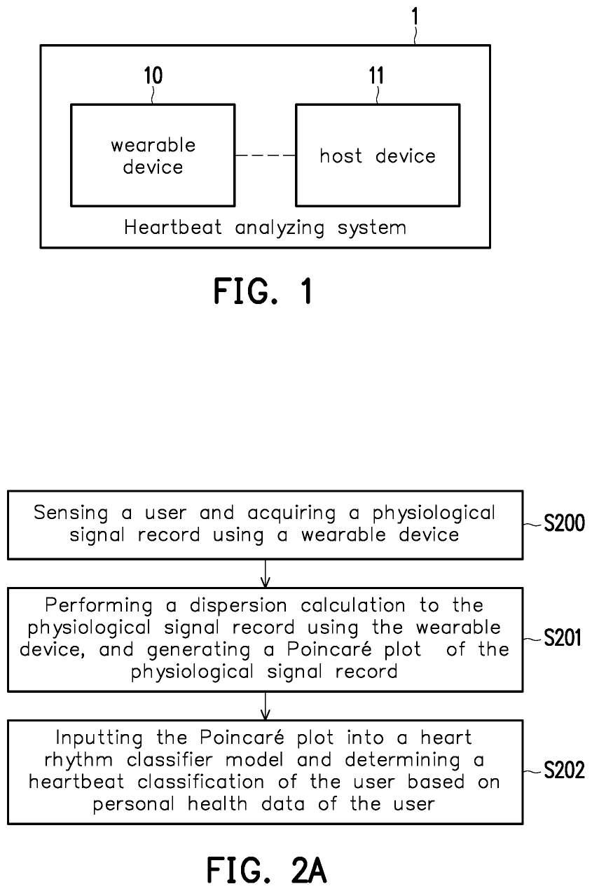

FIG. 1 is a schematic view of a heartbeat analyzing system 1 according to an embodiment of the disclosure. The heartbeat analyzing system 1 includes a wearable device 10 and a host device 11. The wearable device 10 senses a physiological signal record of a user and performs operations, such as filtering, sampling, and segmentation, to the acquired physiological signal record to acquire a heartbeat signal of the user, and then generates accordingly a Poincaré plot of the heartbeat signal. In addition, the wearable device 10 may perform a dispersion calculation to the physiological signal record and the Poincaré plot to acquire the dispersion in relation to the physiological signal record. The host device 11 is communicatively connected to the wearable device 10 and may generate the Poincaré plot and the dispersion of the physiological signal record of the user. In addition, the host device 11 stores a heart rhythm classifier model, and may store or acquire the personal health data of the user. By doing so, the host device 11 may input the Poincaré plot and the dispersion of the physiological signal record of the user into the heart rhythm classifier model, and classify the physiological signal record of the user based on the personal health data of the user and determine whether the heartbeat of the user is regular or not.

On the whole, after acquiring the physiological signal record of the user using the wearable device 10, the heartbeat analyzing system 1 may generate the corresponding Poincaré plot and the dispersion. After generating the Poincaré plot and the dispersion of the physiological signal record, the host device 11 may input the same into the heart rhythm classifier model stored in the host device 11, and classify the physiological signal record based on the personal health data of the user and further determine whether the heartbeat of the user is regular or not. In brief, the heartbeat analysis system 1 may perform the dispersion calculation to the physiological signal records using the wearable device 10, and based on the personal health data of the user, the wearable device 10 may further perform a personalized deep-learning determination based on the physiological signal record to determine whether the heartbeat of the user is regular or not.

Specifically speaking, the wearable device 10 may be, for example, a smart watch, a smart wristband, a pair of smart glasses, and other device of the likes, which may acquire the physiological signal record by being worn on the user's body. Alternatively, the wearable device 10 may also be a personal digital assistant (PDA), a smart phone, a mobile device, a scanner, a camera, a wireless sensor, and other devices of the likes, which are convenient to be carried and may sense the physiological signal record of the user and perform the calculation to the physiological signal record of the user to generate the Poincaré plot and the dispersion. The wearable device 10 may acquire the physiological signal record of the user in all kinds of suitable ways. For example, the wearable device 10 may acquire signals of electrocardiography (ECG), photoplethysmography (PPG), stethoscope, or other physiological signal record containing the heart rhythm or heartbeat of the user as long as the physiological signal record acquired by the wearable device 10 contains features such as heartbeat intervals or heartbeat waves of the user.

The host device 11 may be, for example, a workstation, an advanced mobile station (AMS), a server, a client, a desktop computer, a notebook computer, a network computer, a personal digital assistant (PDA), a personal computer (PC), a tablet computer, etc., which may store the heart rhythm classifier model and the personal health data of the user, in order to perform the personalized deep-learning determination based on the physiological signal record. The personal health data stored on the host device 11 may include, for example, at least one of a medical record, a vital sign, and a medical image of the user. Furthermore, the medical record may include, for example, the user's sex, age, height, weight, body mass index (BMI), body surface area, data of past medical history, medication history, family medical history, current medication status, etc. The vital sign may include, for example, blood pressure, heartbeat, heart rhythm, respiratory rate, oxygen saturation level (OSL), body temperature, pain index, etc. The medical image may include, for example, the user's images of echocardiography, X-ray, nuclear magnetic resonance imaging (NMRI), etc. as long as it contains or may be adapted to determine the user's heart size, left/right atrium size, or left ventricular ejection rate (LVEF).

The wearable device 10 may be connected to the host device 11 in a wired or wireless way. For example, the wireless/wired connections between the wearable device 10 and the host device 11 may be a wireless fidelity (WiFi) communication interface, a Bluetooth communication interface, an infrared radiation (IR) communication interface, a ZigBee communication interface, and/or other wireless communication interfaces, a local area network (LAN) interface, and a universal serial bus (USB) interface, etc.

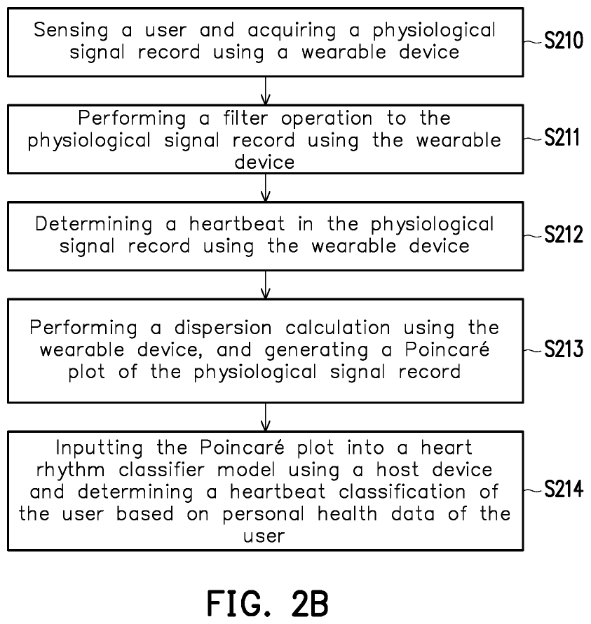

Please then refer to FIG. 2A and FIG. 2B. FIG. 2A is a schematic view of a heartbeat analyzing method according to an embodiment of the disclosure. FIG. 2B is a schematic view of a heartbeat analyzing method according to another embodiment of the disclosure. The heartbeat analyzing methods illustrated in FIG. 2A and FIG. 2B may be executed by the heartbeat analyzing system 1 illustrated in FIG. 1. In the embodiment illustrated in FIG. 2A, the heartbeat analyzing method includes steps S200 to S202. In step S200, the wearable device 10 senses the user and acquires the physiological signal record. In step S201, the wearable device 10 performs the dispersion calculation to the physiological signal record and generates the Poincaré plot of the physiological signal record. In step S202, the Poincaré plot is input into the heart rhythm classifier model and the heartbeat classification of the user is determined based on the personal health data of the user.

In the embodiment illustrated in FIG. 2B, the heartbeat analyzing method includes steps S210 to S215. In step S210, the wearable device 10 senses the user and acquires the physiological signal record. In step S211, the wearable device 10 performs a filter operation to the physiological signal record. In step S212, the wearable device 10 determines the heartbeat in the physiological signal record. In step S213, the wearable device 10 performs the dispersion calculation and generates the Poincaré plot of the physiological signal record. In step S214, the host device 11 inputs the Poincaré plot into the heart rhythm classifier model and determines the heartbeat classification of the user based on the personal health data of the user.

Specifically speaking, in step S210, the wearable device 10 acquires the physiological signal record which includes the heart rhythm or heartbeat of the user. In step S211, the wearable device 10 performs filtering, data cleaning, or detrending to the physiological signal record to filter out noise, errors, or signal deviation values in the signal, and acquires preferably the ideal signal record of the user's heartbeat or heart rhythm which is more suitable for analysis. In step S211, the wearable device 10 determines the heartthrob or heartbeat in the physiological signal record. For example, the wearable device 10 may determine the heartthrob or heartbeat in the physiological signal record through the automatic multiscale-based peak detection (AMPD) algorithm, Pan-Tomkins algorithm, or other algorithms suitable for determining the heartthrob of heartbeat in the physiological signal record, and the wearable device 10 may thereby determine the heartbeat or heartthrob interval in the physiological signal record. In addition, the wearable device 10 may further perform segmentation to the physiological signal record to acquire the physiological signal record having a preset length of time. For example, the preset length of time may be 30 seconds, 45 seconds, 60 seconds, 90 seconds, etc. Furthermore, the wearable device 10 may change the timing of segmentation based on the requirement in need. For example, the wearable device 10 may perform segmentation before the filtering operation of step S211. Or, the wearable device 10 may perform segmentation after the filtering operation of step S211 and before the determination of the heartbeat of step S212. Or, the wearable device 10 may perform segmentation after the determination of the heartbeat of step S212.

In step S213, the wearable device 10 first generates the Poincaré plot of the physiological signal record. The Poincaré plot is a graph points with the length of time of heartbeat or heartthrob interval on horizontal axis versus the length of time of succeeding heartbeat or heartthrob interval on vertical axis. Therefore, the Poincaré plot may be seen as the visualization of oscillations of heartbeat or heartthrob interval in time unit in the physiological signal record. Then, the wearable device 10 calculates the dispersion of the physiological signal record based on the Poincaré plot. More specifically, the dispersion calculated by the wearable device 10 is a ratio, the numerator of the ratio is the standard deviation of distances from multiple points in the Poincaré plot to the diagonal (that is, the y=x straight line of the Poincaré plot), and the denominator of the ratio is the vertical or horizontal coordinate value of the point on the diagonal that minimizes the sum of distances from each point in the Poincaré plot to it. Therefore, the dispersion may be seen as the ratio further calculated by adapting the oscillations of heartbeat or heartthrob interval in time unit in the physiological signal record.

In step S214, the host device 11 may receive the Poincaré plot and the dispersion provided by the wearable device 10. The storage (not illustrated in FIG. 1) of the host device 11 stores the heart rhythm classifier model. The host device 11 may input the received Poincaré plot and the dispersion into the heart rhythm classifier model to determine the heartbeat classification of the user through machine learning, and further determine whether the heartbeat of the user is regular or arrhythmic. More specifically, the heart rhythm classifier model is a model generated by training beforehand using a plurality of heartbeat signal records that have been marked to be regular or arrhythmic. By inputting the physiological signal record into the heart rhythm classifier model, the host device 11 may determine whether the heart rhythm of the user is regular or arrhythmic. In addition, the storage of the host device 11 also stores the personal health data of the user, as all kinds of health conditions of the user may be adopted to assist in the determination of the heartbeat classification of the user. For example, the personal health data includes at least one of the medical record, the vital sign, and the medical image. Therefore, when the heartbeat classification is to be determined, with the help of the personal health data, the host device 11 may consider preferably the heart-related parameters captured from the user's past medication history, personal physical condition, and medical images to assist in the determination, thereby eliminating the non-ideal factors in determining the heartbeat classification of the user and determining accurately the heartbeat classification of the physiological signal record based on the personal health data of the user. In order for the heart rhythm classifier model to make accurate determination based on the personal health data, when the heart rhythm classifier model is trained beforehand, the input heartbeat signal records which have been marked must include accordingly the corresponding health data for the heart rhythm classifier model to find out the correlation between the personal health data and the heartbeat classification. For example, the heartbeat classification determined by the host device 11 may be comet-shaped, torpedo-shaped, fan-shaped, and complex. Accordingly, the host device 11 may determine the heart rhythm of the user to be regular or arrhythmic.

Therefore, the heartbeat analyzing method may acquire the Poincaré plot and the dispersion of the physiological signal record using the wearable device. By means of machine learning, the host device determines the heartbeat classification of the physiological signal record. The host device may further take into consideration the personal health data of the user as factors to assist in the determination of the heartbeat classification. By doing so, the heartbeat analyzing method may determine accurately the heartbeat classification of the user and further determine the heart rhythm of the user to be regular or arrhythmic.

In sum of the above, the heartbeat analyzing method and heartbeat analyzing device of the disclosure may acquire the physiological signal record of the user to generate the Poincaré plot and the dispersion. The heart rhythm classifier model performs machine learning based on the personal health data of the user, and thereby makes a personalized determination based on the personal physiological signal record of the user, therefore lowering the possibility of making human errors and further improving efficiently the accuracy in determining whether a heart rhythm is regular or not.

What is claimed is:

1. A heartbeat analyzing method, comprising:
sensing a user using a wearable device to acquire a physiological signal record;
performing a dispersion calculation to the physiological signal record using the wearable device to generate a Poincaré plot of the physiological signal record; and
inputting the Poincaré plot into a heart rhythm classifier model to determine a heartbeat classification of the user based on personal health data of the user.

2. The heartbeat analyzing method according to claim 1, further comprising performing a filter operation to the physiological signal record using the wearable device.

3. The heartbeat analyzing method according to claim 1, further comprising determining a plurality of wave crests in the physiological signal record and a plurality of time intervals between the wave crests using the wearable device.

4. The heartbeat analyzing method according to claim 1, wherein performing the dispersion calculation to the physiological signal record using the wearable device further comprises:
performing segmentation to the physiological signal record and acquiring a physiological signal record segment having a preset length of time.

5. The heartbeat analyzing method according to claim 1, wherein the personal health data comprises at least one of a medical record, a vital sign, and a medical image of the user.

6. The heartbeat analyzing method according to claim 1, wherein the heart rhythm classifier model is generated by training beforehand using machine learning, and that inputting the Poincaré plot and the personal health data of the user into the heart rhythm classifier model to determine the heartbeat classification of the user comprises:
classifying a pattern of the Poincaré plot based on the personal health data using the heart rhythm classifier model, and determining the heartbeat classification of the user.

7. The heartbeat analyzing method according to claim 1, wherein the dispersion calculation corresponds to a ratio of a numerator to a denominator, the numerator is a standard deviation of a plurality of distances from a plurality of points in the Poincaré plot to a diagonal, and the denominator is a corresponding coordinate value of a second point on the diagonal, wherein there is a minimum of sum of a plurality of second distances from the points in the Poincaré plot to the second point.

8. The heartbeat analyzing method according to claim 7, wherein the diagonal is a y=x straight line corresponding to the Poincaré plot.

9. A heartbeat analyzing system, comprising:
a wearable device, sensing a user to acquire a physiological signal record and performing a dispersion calculation to the physiological signal record and generating a Poincaré plot of the physiological signal record; and
a host device, being communicatively connected to the wearable device and storing a heart rhythm classifier model, the host device inputting the Poincaré plot and personal health data of the user into the heart rhythm classifier model and determining a heartbeat classification of the user.

10. The heartbeat analyzing system according to claim 9, wherein the dispersion calculation performed by the host device is to calculate a ratio of a numerator to a denominator, the numerator is a standard deviation of a plurality of distances from a plurality of points in the Poincaré plot to a diagonal, and the denominator is a corresponding coordinate value of a second point on the diagonal, wherein there is a minimum of sum of a plurality of second distances from the points in the Poincaré plot to the second point.
